(19) United States
(12) Patent Application Publication
(10) Pub. No.: US 2003/0191390 A1
(43) Pub. Date: Oct. 9, 2003

(54) Ultrasonic treatment apparatus

(75) Inventor: Eiji Murakami, Hachioji-shi (JP)

Correspondence Address: OSTROLENK FABER GERB & SOFFEN, 1180 AVENUE OF THE AMERICAS, NEW YORK, NY, 100368403

(73) Assignee: Olympus Optical Co., Ltd.

(21) Appl. No.: 10/408,940

(22) Filed: Apr. 9, 2003

(30) Foreign Application Priority Data
Apr. 9, 2002 (JP) 2002-106840

(51) Int. Cl. A61B 8/00

(52) U.S. Cl. 600/439

(57) ABSTRACT

An ultrasonic instrument and an ultrasonic cleaner share an ultrasonic treatment apparatus body that is connected electrically to a transducer unit of the instrument and outputs electric information for generating ultrasonic waves. The apparatus body also delivers electric information for generating ultrasonic waves to the ultrasonic cleaner.

### CROSS-REFERENCE TO RELATED APPLICATIONS

This application is based upon and claims the benefit of priority from the prior Japanese Patent Application No. 2002-106840, filed Apr. 9, 2002, the entire contents of which are incorporated herein by reference.

### BACKGROUND OF THE INVENTION

1. Field of the Invention

The present invention relates to an ultrasonic treatment apparatus for surgical operation, capable of carrying out treatment, such as incision, ablation, or coagulation of organic tissue, by means of ultrasonic waves.

2. Description of the Related Art

Ultrasonic treatment apparatuses have conventionally been used to carry out treatment, such as incision, ablation, or coagulation of organic tissue, by means of ultrasonic waves. One such apparatus is described in Jpn. Pat. Appln. KOKAI Publication No. 2000-271135, for example.

As shown in FIG. 9, this ultrasonic treatment apparatus comprises an ultrasonic instrument a, which is manually operated by an operator, and an apparatus body c connected to the instrument a by means of a connecting cord b. The ultrasonic treatment apparatus body c is electric information output means that outputs electric information for generating ultrasonic vibration. It is set in an operating room, for example.

In the ultrasonic instrument a, a control section e on the hand side is coupled to the proximal end portion of an elongate insert section cannula d. The control section e is provided with an ultrasonic transducer f that generates ultrasonic vibration. Further, a treatment section g for treating organic tissue is disposed on the-distal end portion of the cannula d.

A vibration transmitting member h is passed through the insert section cannula d. The proximal end portion of the member h is connected to the ultrasonic transducer f. Further, an ultrasonic probe hi on the side of the treatment section g is formed on the distal end portion of the member h. When the ultrasonic treatment apparatus is driven, the ultrasonic vibration generated by the ultrasonic transducer f is transmitted to the ultrasonic probe h1 on the side of the treatment section g through the vibration transmitting member h.

A jaw i is disposed on the treatment section g so as to face the probe h1. The proximal end portion of the jaw i is rotatably supported on the distal end portion of the insert section cannula d. A grasping member portion of the jaw i that touches the organic tissue is formed of a resin material such as polytetrafluoroethylene.

The control section e is provided with a control handle j that can open and close the jaw i with respect to the ultrasonic probe h1. Further, a control rod (not shown) for transmitting control force to open and close the jaw i is inserted in the insert section cannula d.

When the ultrasonic treatment apparatus is in operation, the control rod is moved in the axial direction as the control handle j is manipulated. As the control rod is moved in this manner, the jaw i of the treatment section g is opened or closed with respect to the ultrasonic probe h1. If the jaw i is then closed, the organic tissue can be held between the probe h1 and the jaw i. Subsequently, in this state, the ultrasonic vibration from the ultrasonic transducer f is transmitted to the ultrasonic probe h1 on the side of the treatment section g through the vibration transmitting member h. Thus, the organic tissue can be subjected to treatment such as incision, ablation, or coagulation, by means of ultrasonic waves.

An ultrasonic treatment system that also utilizes ultrasonic waves is described in Jpn. Pat. Appln. KOKAI Publication No. 2000-296132. In this ultrasonic treatment system, only one power source unit can serve to control various other applications for ultrasonic treatment than the aforesaid ultrasonic instrument that has a tip working portion. These other applications include an ultrasonic instrument that has functions for water supply and suction, for example, and serves to carry out treatment such as fragmentation, emulsification, or coagulation of organic tissue. Another example is an ultrasonic instrument that has a trocar function to bore a treatment hole for endoscopic surgical operation in a patient's body.

Further, an ultrasonic cleaner is a known means for ultrasonically cleaning an instrument. It ultrasonically cleans the instrument that is soiled as it is used for medical treatment of organic tissue. In operating the ultrasonic cleaner, the instrument is put into a cleaning tank that is filled with a cleaning fluid, and ultrasonic waves are generated in the tank. By doing this, dirt on the instrument can be removed.

In the ultrasonic treatment apparatus, the treatment section g of the ultrasonic instrument a is expected to have an optimum shape that matches the treatment region of organic tissue, as an object of ultrasonic treatment, and the treatment method. In an instrument for endoscopic surgical operation, in particular, the outside diameter of its insert section should be minimized to lessen invasion. Accordingly, the treatment section g at the distal end portion is thinned and reduced in size, and is composed of a precision part. Thus, dirt, such as blood or another body fluid, easily adheres to the treatment section g at the distal end portion of the ultrasonic instrument a during operation.

After the operation, therefore, the treatment section g at the distal end of the ultrasonic instrument a, in particular, must be cleaned satisfactorily. Conventionally, a brush or other cleaning tool is used for this cleaning operation.

Conventionally, moreover, an operating theater is not expected to be furnished with a cleaner. Usually, therefore, instruments are cleaned in another room.

### BRIEF SUMMARY OF THE INVENTION

According to the present invention, there is provided an ultrasonic treatment apparatus comprising: an ultrasonic instrument which has ultrasonic vibration generating means for generating ultrasonic vibration and treats organic tissue with the ultrasonic vibration generated by the ultrasonic vibration generating means; ultrasonic cleaning means for cleaning a medical appliance by means of ultrasonic vibration; and electric information output means for delivering electric information for generating ultrasonic vibration to the ultrasonic vibration generating means and/or the ultrasonic cleaning means.

According to the invention, moreover, there is provided an ultrasonic treatment apparatus comprising: an application for ultrasonic treatment; and a cleaning unit for cleaning an operating apparatus by utilizing ultrasonic vibration, the application comprising a transducer, a power source which outputs electric information or signal to cause the transducer to generate ultrasonic vibration, and a probe which has a proximal end portion removably attached to the transducer and a distal end portion fitted with a tip treatment portion for treating organic tissue and transmits the ultrasonic vibration from the transducer to the tip treatment portion.

According to the invention, the ultrasonic instrument and the ultrasonic cleaning means share the electric information output means that is connected electrically to the ultrasonic vibration generating means of the instrument and outputs electric information for generating ultrasonic waves. The electric information output means also delivers electric information for generating ultrasonic waves to the cleaning means. The medical appliance to be cleaned by means of the cleaning means is not limited to the ultrasonic instrument, and may alternatively be an electric knife, grasping forceps, or any other soiled instrument that can be ultrasonically cleaned.

Additional objects and advantages of the invention will be set forth in the description-which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate presently preferred embodiments of the invention, and together with the general description given above and the detailed description of the preferred embodiments given below, serve to explain the principles of the invention.

FIG. 1 is a perspective view showing a configuration of the entire system of an ultrasonic treatment apparatus according to a first embodiment of the invention;

FIG. 2 is a side view showing an external appearance of the entire ultrasonic treatment apparatus of the first embodiment;

FIG. 3 is a side view showing a disassembled state of the ultrasonic treatment apparatus of the first embodiment;

FIG. 4 is a longitudinal sectional view of a principal part showing a detailed configuration of the distal end portion of a handle unit of the ultrasonic treatment apparatus of the first embodiment;

FIG. 5 is a side view showing the way the distal end portion of a handle unit of an ultrasonic treatment apparatus according to a second embodiment of the invention is inserted and cleaned in a cleaning unit;

FIG. 6A is a longitudinal sectional view of a principal part showing the state that the distal end portion of the handle unit of the ultrasonic treatment apparatus of the second embodiment is cleaned;

FIG. 6B is a cross-sectional view taken along line VIB-VIB of FIG. 6A;

FIG. 7 is a side view showing an external appearance of a cleaning probe according to a third embodiment of the invention;

FIG. 8 is a longitudinal sectional view of a principal part showing the way a handle unit of the third embodiment, having the cleaning probe joined thereto, is cleaned in a cleaning tank; and

FIG. 9 is a perspective view showing an external appearance of the entire system of a conventional ultrasonic treatment apparatus.

### DETAILED DESCRIPTION OF THE INVENTION

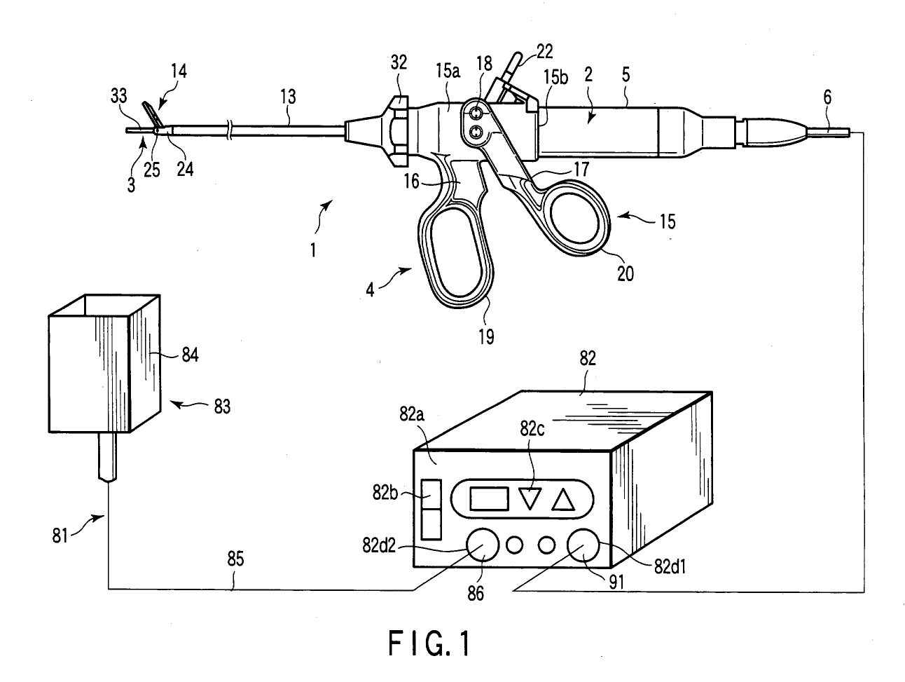

A first embodiment of the present invention will now be described with reference to FIGS. 1 to 4. FIG. 1 shows a configuration of the entire system of an ultrasonic treatment apparatus 81 of the present embodiment. The apparatus 81 comprises an ultrasonic treatment apparatus body (electric information output means) 82, an ultrasonic instrument 1 for surgical operation, and an ultrasonic cleaner (ultrasonic cleaning means) 83.

Further, a front panel 82a of the ultrasonic treatment apparatus body 82 is provided with a power switch 82b, a display section 82c, and a plurality of connector receiving portions, i.e., portions 82d1 and 82d2 according to the present embodiment.

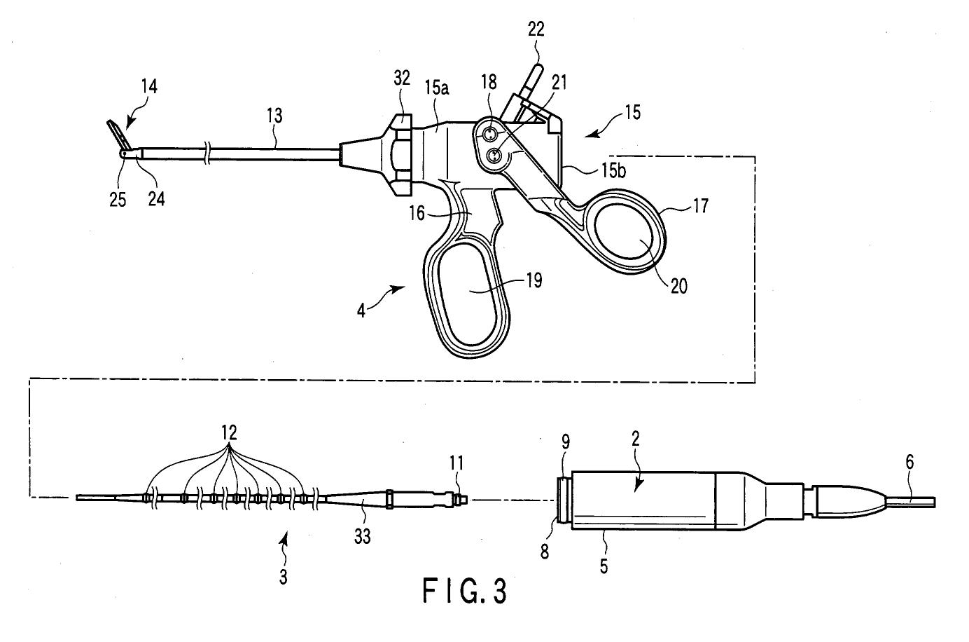

As shown in FIG. 3, moreover, the ultrasonic instrument 1 is composed of three units, a transducer unit (ultrasonic vibration generating means) 2, a probe unit 3 for treatment, and a handle unit 4, which can be individually disjoined from and joined to one another.

A piezoelectric element (a transducer) not shown, that converts current into ultrasonic vibration is incorporated in the transducer unit 2. The outside of the piezoelectric element is covered by a substantially cylindrical transducer cover 5.

Further, one end portion of a cord 6 is coupled to the rear end of the transducer unit 2. A joint connector 91 is coupled to the other end portion of the cord 6. The joint connector 91 is removably connected to the one or first connector receiving portion 82d1 of the ultrasonic treatment apparatus body 82. Current for generating ultrasonic vibration delivered from the apparatus body 82 is supplied to the transducer unit 2 by means of the cord 6.

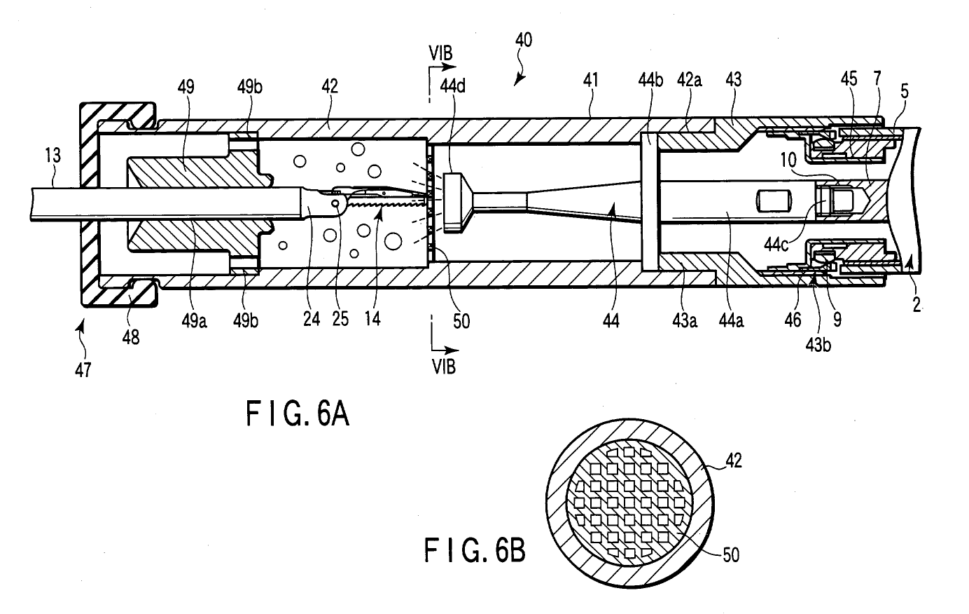

A horn 7 (shown in FIG. 6A) is located on the distal end side of the transducer unit 2. The proximal end portion of the horn 7 is attached to the piezoelectric element. As shown in FIG. 3, moreover, an attachment 8 for the connection of the handle unit 4 is fixed to the distal end portion of the transducer cover 5. The attachment 8 is fitted with a metallic engaging ring 9, a part of which is cut.

As shown in FIG. 6A, the distal end portion of the horn 7 is provided with a tapped hole portion 10 to which the probe unit 3 for treatment can be attached. As shown in FIG. 3, the proximal end portion of the probe unit 3 is provided with an external thread portion 11 that is screwed into the threaded hole portion 10 of the horn 7. The male screw portion 11 of the probe unit 3 is screwed into the tapped hole portion 10 of the horn 7, thereby, the transducer unit 2 and the probe unit 3 are connected to each other. In this state, ultrasonic vibration from a transducer of the transducer unit 2 is transmitted to the probe unit 3 through the horn 7.

The probe unit 3 for treatment is provided with a treatment probe (vibration transmitting member) 33 in the form of an elongate rod. The probe 33 is designed so that its overall length is equal to an integral multiple of the half-wave length of the ultrasonic vibration.

Further, the treatment probe 33 has vibration nodes that are arranged in a plurality of positions along its axis. Amplitude adjusting portions are arranged corresponding to a plurality of vibration nodes in the middle of the probe 33. They can adjust the amplitude by reducing the axial cross section, so that the distal end of the probe 33 obtains the required amplitude. The treatment probe 33 is fitted with rubber rings 12, which are arranged corresponding to several vibration nodes in the middle. The rings 12 serve to prevent internal parts (mentioned later) of the handle unit 4 from interfering with the probe unit 3.

Further, the handle unit 4 is provided with an insert section 13 in the form of an elongate pipe. In a surgical operation, the insert section 13 is inserted into a patient's body cavity. A tip working portion (manipulating portion) 14 is attached to the distal end of the insert section 13. A control section 15 for operating the working portion 14 is attached to the proximal end portion of the insert section 13.

The control section 15 is provided with a substantially cylindrical control section body 15a. A stationary handle 16 is formed integrally on the body 15a. Further, a movable handle 17 is rockably mounted on the body 15a by means of a pivot pin 18. The stationary handle 16 and the movable handle 17 are provided with finger loop portions 19 and 20, respectively. The movable handle 17 can be rotated around the pin 18 toward or away from the stationary handle 16 as the handles 16 and 17 are held and moved with fingers in the loop portions 19 and 20.

Furthermore, a working pin 21 is mounted on the movable handle 17. The inner end portion of the pin 21 is in engagement with a driving force transmitting member (not shown) that is located in the control section body 15a.

A transducer unit coupling portion 15b is attached to the proximal end portion of the control section body 15a. The coupling portion 15b is provided with a ring receiving member (not shown) that can releasably engage the engaging ring 9 of the transducer unit 2. If the transducer unit 2 is joined to the transducer unit coupling portion 15b of the control section body 15a, its engaging ring 9 is caused removably to engage the ring receiving member of the control section body 15a by its own elastic force.

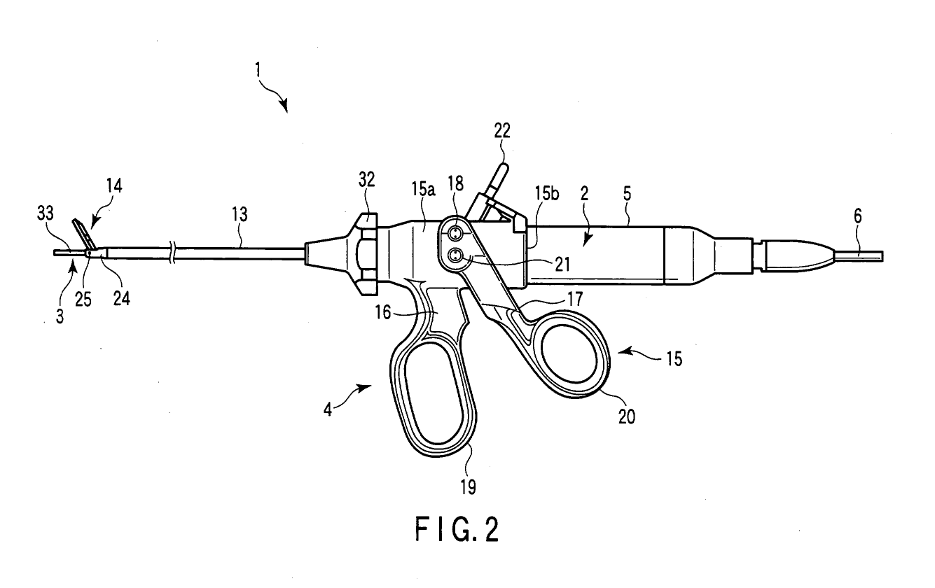

In the handle unit 4 of the ultrasonic instrument 1, as shown in FIG. 2, moreover, a high-frequency connecting pin 22 protrudes from the outer peripheral surface of the proximal end portion of the control section body 15a. The inner end portion of the pin 22 is connected electrically to the probe unit 3 for treatment at the time of assembly. Further, one end portion of a cord for high-frequency current supply is connected to the outer end portion of the pin 22. High-frequency current is supplied to the probe unit 3 through the pin 22, whereby coagulation, incision, or other treatment can be executed. Any component that is exposed to the outside of the control section 15 is formed of an electrical insulating member.

Further, the distal end portion of the control section 15 is provided with a rotary knob 32 for rotating the insert section 13 around its axis. The insert section 13 is attached to the control section 15 in a manner such that it can be rotated around the axis by turning the knob 32.

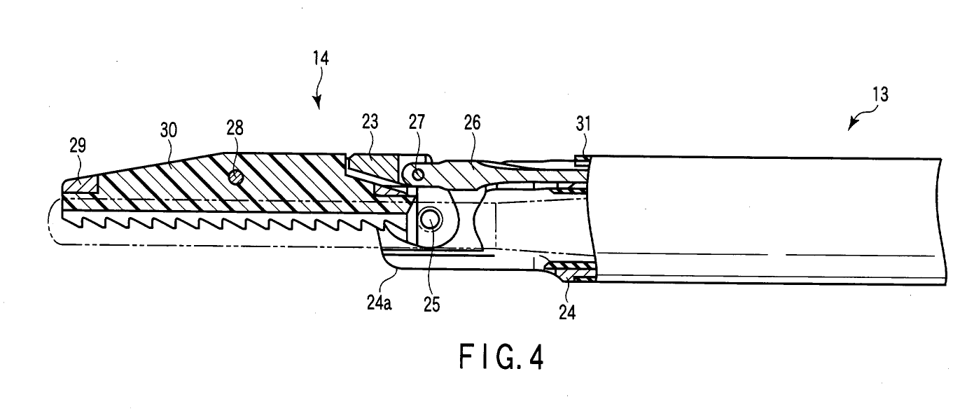

As shown in FIG. 4, moreover, a jaw 23 is attached to the tip working portion 14 on the distal end of the insert section 13. The jaw 23 can be opened and closed with respect to the distal end portion of the treatment probe 33. A substantially cylindrical jaw mounting member 24 is fixed to the distal end of the insert section 13. The jaw mounting member 24 is provided with forked jaw supporting members 24a that extend forward. A pivot pin 25 for the jaw 23 is bridged between the supporting members 24a. The proximal end portion of the jaw 23 is rotatably supported on the mounting member 24 by means of the pin 25.

Furthermore, the distal end portion of a drive shaft 26 is coupled to the proximal end portion of the jaw 23 by means of a pin 27. The proximal end portion of the shaft 26 extends through the interior of the insert section 13 to the control section 15. It is coupled to the driving force transmitting member (not shown) in the control section 15.

Further, the jaw 23 is provided with a grasping portion 30, which can grasp organic tissue in conjunction with the probe unit 3, and a grasping portion mounting member 29 that holds the grasping portion 30. The grasping portion 30 is formed of a low-friction resin material (e.g., polytetrafluoroethylene). The grasping portion mounting member 29 is mounted on the jaw 23 by means of a pin 28 so as to be rotated for a fixed angle. An insulating tube 31 covers the whole outer peripheral surface of the insert section 13.

The ultrasonic cleaner 83 is provided with a cleaning tank 84 in which a medical appliance is ultrasonically cleaned. The tank 84 contains an ultrasonic vibration generating portion (not shown) therein. One end portion of a connecting cord 85 is connected to the ultrasonic vibration generating portion. A joint connector 86 is coupled to the other end portion of the cord 85. The connector 86 is detachably connected to the other or second connector receiving portion 82d2 of the ultrasonic treatment apparatus body 82. Current for generating ultrasonic vibration delivered from the apparatus body 82 is supplied to the ultrasonic cleaner 83 by means of the connecting cord 85. Thus, the medical appliance in the cleaning tank 84 can be cleaned by ultrasonic vibration.

The medical appliance to be cleaned in the cleaning tank 84 is not limited to the ultrasonic instrument 1, and may alternatively be an electric knife, grasping forceps, or any other soiled instrument that can be ultrasonically cleaned. Thus, the ultrasonic instrument 1 and the ultrasonic cleaner 83 share the one ultrasonic treatment apparatus body 82 with each other.

The following is a description of the operation of the apparatus constructed in this manner. Before the ultrasonic treatment apparatus 81 of the present embodiment is worked, the three units of the ultrasonic instrument 1, including the transducer unit 2, probe unit 3 for treatment, and handle unit 4, are joined to one another, as shown in FIG. 1.

In this state, the joint connector 91 of the ultrasonic instrument 1 is detachably connected to the first connector receiving portion 82d1 of the ultrasonic treatment apparatus body 82. Further, the joint connector 86 of ultrasonic cleaner 83 is detachably connected to the second connector receiving portion 82d2 of the apparatus body 82.

When the ultrasonic instrument 1 is in operation, moreover, the working pin 21 advances or retreats substantially along the axis of insertion if the movable handle 17 is worked. As this is done, driving force is transmitted to the drive shaft 26 through the driving force transmitting member, so that the jaw 23 is rotated around the pivot pin 25. Thereupon, the jaw 23 is opened or closed with respect to the tip manipulating portion of the treatment probe 33 of the probe unit 3 for treatment. Thus, the organic tissue can be held between the jaw 23 and the distal end portion of the probe 33.

When the organic tissue is held between the jaw 23 and the distal end portion of the treatment probe 33, the grasping portion mounting member 29 of the jaw 23 and the grasping portion 30 rotate around the pin 28, following the deflection of the probe unit 3 for treatment. Thus, the organic tissue can be held with uniform force between the jaw 23 and the distal end portion of the treatment probe 33.

In the ultrasonic instrument 1 of the present embodiment, thereafter, the transducer of the transducer unit 2 is driven with the organic tissue held between the jaw 23 and the distal end portion of the treatment probe 33. As this is done, the ultrasonic waves are generated by means of the transducer of the transducer unit 2, whereupon ultrasonic vibration is transmitted from the transducer to the probe unit 3 for treatment through the horn 7. Thus, the organic tissue between the jaw 23 and the distal end portion of the treatment probe 33 is subjected to treatment such as coagulation, incision, etc.

Further, a cord for high-frequency current supply is connected to the high-frequency connecting pin 22 of the ultrasonic instrument 1 so that high-frequency current can be supplied from a high-frequency cauterization power source unit. Thus, the high-frequency current is supplied to the probe unit 3 for treatment through the pin 22, whereby the organic tissue can be subjected to high-frequency treatment by means of the distal end portion of the probe unit 3.

The dirty medical appliance such as a used ultrasonic instrument 1 is inserted in the cleaning tank 84 of the ultrasonic cleaner 83. The cleaner 83 is driven in this state. As this is done, the current for generating the ultrasonic vibration delivered from the ultrasonic treatment apparatus body 82 is supplied to the cleaner 83 by means of the connecting cord 85. Thus, the ultrasonic vibration can be used to clean the medical appliance in the tank 84.

The configuration described above has the following effects. The ultrasonic treatment apparatus 81 of the present embodiment shares with the ultrasonic cleaner 83 the ultrasonic treatment apparatus body 82 that is connected electrically to the ultrasonic transducer of the ultrasonic instrument 1 and outputs electric information for generating ultrasonic waves. The apparatus body 82 can also deliver the electric information for generating ultrasonic waves to the cleaner 83. Therefore, components of the ultrasonic cleaner 83 other than the cleaning tank 84 need not be newly arranged in an operating room, so that the cleaner 83 can be used in a narrow space in the room. In consequence, dirt on the tip working portion 14 of the ultrasonic instrument 1 can be quickly removed by ultrasonic cleaning by means of the ultrasonic cleaner 83 before it dries, during a surgical operation. Thus, dirt on the working portion 14 can be removed more efficiently, and in less time than in the case where it is removed by ultrasonic cleaning after a surgical operation.

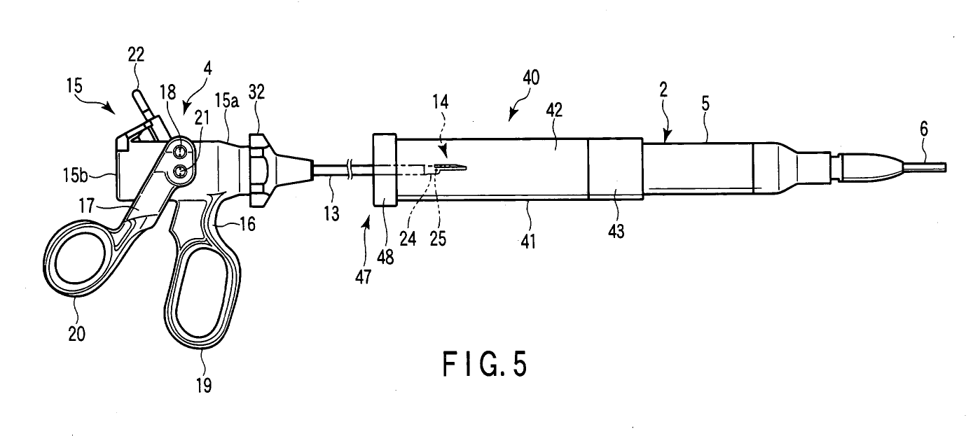

FIGS. 5, 6A and 6B show a second embodiment of the invention. According to the present embodiment, the configuration of the ultrasonic treatment apparatus 81 of the first embodiment (see FIGS. 1 to 4) is modified in the following manner.

As shown in FIG. 5, the ultrasonic treatment apparatus 81 of the present embodiment is provided with a cleaning unit 40 that can clean the tip working portion 14 of the ultrasonic instrument 1 by utilizing the ultrasonic waves generated from the transducer of the instrument 1.

The cleaning unit 40 is furnished with a cleaning tank 41 that can be attached to and detached from the ultrasonic instrument 1. As shown in FIGS. 6A and 6B, the cleaning tank 41 is provided with a cylindrical cleaning unit cover 42. A cylindrical connecting cover 43 is attached to one end portion of the cover 42. A small-diameter coupling cylinder portion 43a is formed on the proximal end portion of the cover 43. It can be inserted into the cleaning unit cover 42. On the other hand, a large-diameter coupling hole portion 42a is formed on the inner peripheral surface of the one end side of the cover 42.

Further, a cleaning probe 44 for transmitting ultrasonic vibration is located on one end side of the cleaning tank 41. The cleaning probe 44 is provided with an elongate probe body 44a. A flange portion 44b that is substantially equal in diameter to the coupling hole portion 42a of the cover 42 is provided substantially in the central part of the probe body 44a. The flange portion 44b is located corresponding to a vibration node of the cleaning probe 44.

When the flange portion 44b of the cleaning probe 44 is in the coupling hole portion 42a of the cleaning unit cover 42, the coupling cylinder portion 43a of the connecting cover 43 can be inserted into the coupling hole portion 42a of the cover 42. Thus, the cleaning probe 44 is mounted in a manner such that its flange portion 44b is held between the cleaning unit cover 42 and the connecting cover 43.

The proximal end portion of the cleaning probe 44 is provided with an external thread portion 44c that is screwed into the threaded hole portion 10 of the horn 7. As the male screw portion 44c of the probe 44 is screwed into the hole portion 10 of the horn 7, the transducer unit 2 and the probe 44 can be removably coupled to each other. In this state, ultrasonic vibration from the transducer of the transducer unit 2 can be transmitted to the probe 44 through the horn 7.

Further, the distal end portion of the cleaning probe 44 is provided with a cavitation portion 44d having a shape such that cavitation can be efficiently caused in a cleaning fluid during ultrasonic oscillation.

Furthermore, a transducer unit coupling portion 43b is provided on the inner peripheral surface side of the distal end portion of the connecting cover 43. The coupling portion 43b is fitted with a guide 45 and a ring receiving member 46, which serve to form a groove that can releasably engage the engaging ring 9 of the transducer unit 2. If the transducer unit 2 is joined to the coupling portion 43b of the cleaning tank 41 of the cleaning unit 40, its engaging ring 9 is caused to removably engage the ring receiving member 46 by its own elastic force.

An inlet portion 47 of the insert section 13 of the ultrasonic instrument 1 is located at the other end portion of the cleaning unit cover 42. A gasket 48 of an elastic material such as rubber is attached to the inlet portion 47. Further, the cleaning tank 41 is filled with the cleaning fluid between the gasket 48 and the flange portion 44b of the probe 44. The gasket 48 serves to prevent the fluid from leaking out as the insert section 13 of the handle unit 4 of the instrument 1 is inserted into the inlet portion 47.

A stabilizer 49 for fixing the insert section 13 of the ultrasonic instrument 1 is attached to the inner peripheral surface of the inlet portion 47 of the cleaning unit cover 42. A passage hole 49a for the insert section 13 penetrates the center portion of the stabilizer 49. Further, a plurality of cleaning fluid circulation holes 49b are formed in the peripheral edge portion of the stabilizer 49.

Furthermore, a net 50 is attached to the middle portion of the cleaning unit cover 42, adjoining the cleaning probe 44. As shown in FIG. 6B, the net 50 is formed having substantially latticed meshes. During a cleaning operation, the net 50 in the middle of the cover 42 serves to prevent the tip working portion 14 of the ultrasonic instrument 1 from directly touching the cleaning probe 44. The net 50 is located in a position such that the working portion 14 can enjoy a good ultrasonic cleaning effect.

The following is a description of the operation of the apparatus constructed in this manner. Before the cleaning unit 40 of the present embodiment is worked, the ultrasonic instrument 1 is disassembled into three units, including the transducer unit 2, probe unit 3 for treatment, and handle unit 4. In this state, the cleaning unit 40 is attached to the transducer unit 2.

After the cleaning tank 41 is filled with the cleaning fluid, the distal end portion of the insert section 13 of the handle unit 4 is passed through the passage hole 49a of the stabilizer 49 via a hole in the center of the gasket 48 so that it runs against the net 50. The transducer of the transducer unit 2 is driven in this state. Ultrasonic waves are then generated by means of the transducer of the transducer unit 2, ultrasonic vibration from the transducer is transmitted to the cleaning probe 44 through the horn 7. Thereupon, the cavitation portion 44d at the distal end of the cleaning probe 44 causes cavitation, whereby the tip working portion 14 of the ultrasonic instrument 1 can be efficiently cleaned just after surgical operation.

The configuration described above has the following effects. In the ultrasonic instrument 1 of the present embodiment, the three units, i.e., the transducer unit 2, probe unit 3, and handle unit 4, are disjoined from one another, the cleaning unit 40 is attached to the transducer unit 2. Thus, ultrasonic energy for treatment that is generated by means of the transducer of the transducer unit 2 can be also used to clean the tip working portion 14 of the instrument 1, which can't be easily cleaned.

During and after surgical operation, therefore, the tip working portion 14 of the ultrasonic instrument 1 can be subjected to pre-cleaning (ultrasonic cleaning) readily and effectively. Thus, those parts which are inaccessible to a brush can be easily cleaned in a short time.

Further, the cleaning unit 40 of the present embodiment uses the ultrasonic energy for treatment that is generated by means of the transducer of the transducer unit 2. In general, a frequency of about 38 kHz or more is used as the optimum frequency of an ultrasonic cleaner for the cleaning of precision parts. This frequency matches the frequency band of the ultrasonic instrument 1 with which the organic tissue is coagulated or incised. Accordingly, the tip working portion 14 of the instrument 1 can be effectively cleaned with use of the cleaning unit 40 of the present embodiment.

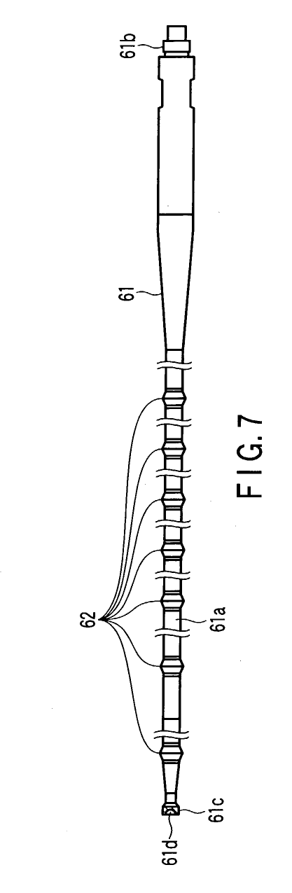

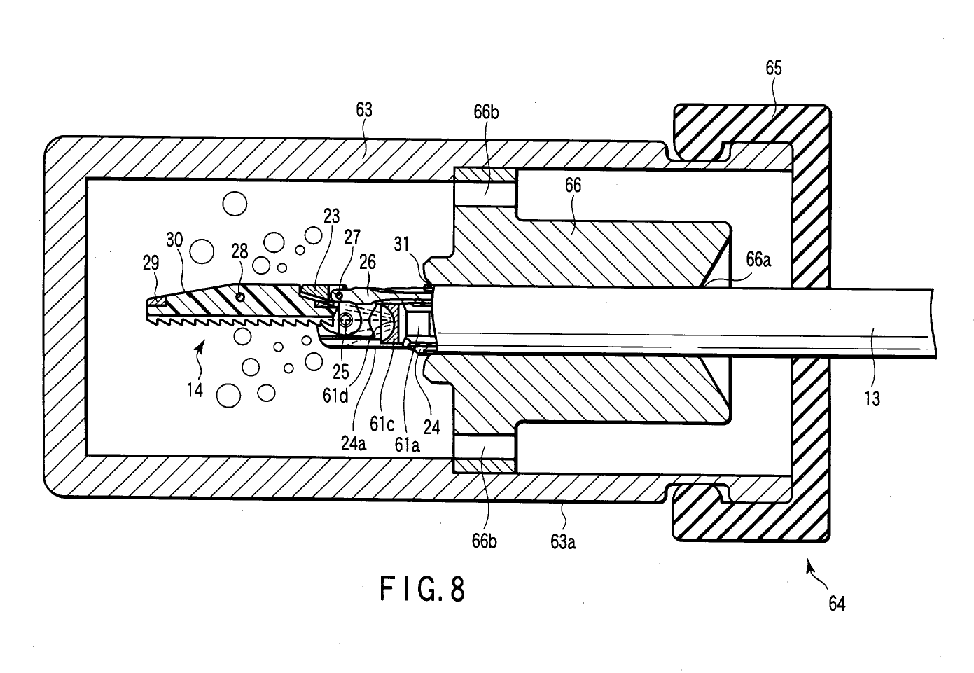

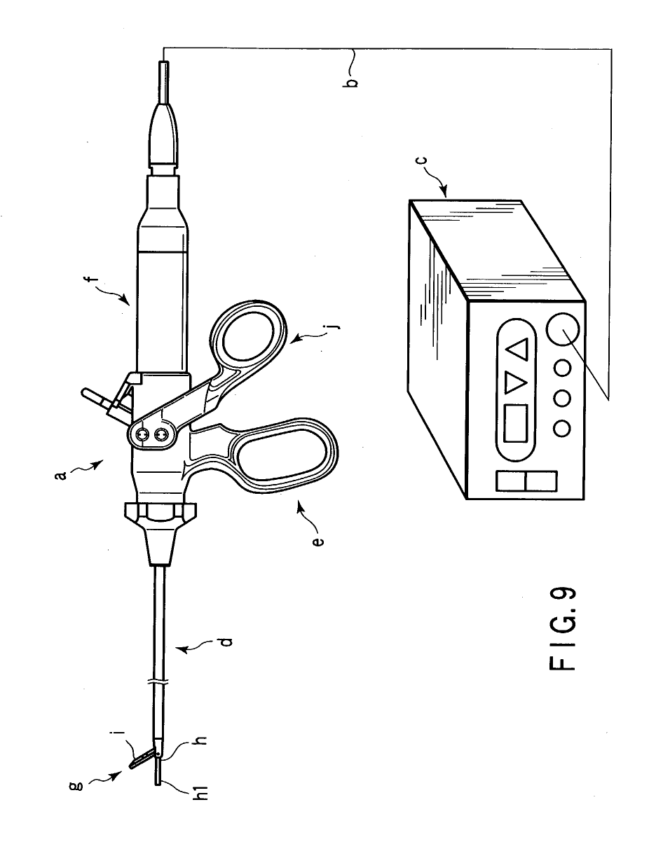

FIGS. 7 and 8 show a third embodiment of the invention. In the present embodiment, a cleaning probe 61 that can be joined to the handle unit 4 is provided in place of the treatment probe 33 of the probe unit 3 of the ultrasonic instrument 1 of the second embodiment (see FIGS. 5, 6A and 6B). The cleaning probe 61 is constructed substantially in the same manner as the treatment probe 33 of the first embodiment, and differs from the latter only in the shape of its distal end.

As shown in FIG. 7, the proximal end portion of an elongate probe body 61a of the cleaning probe 61, like that of the treatment probe 33, is provided with an external thread 61b that is screwed into the tapped hole portion 10 of the horn 7 of the transducer unit 2. Further, the distal end portion of the probe 61 is provided with a cavitation portion 61c having a shape such that cavitation can be efficiently caused in the cleaning fluid during ultrasonic oscillation. As shown in FIG. 8, the distal end face of the cavitation portion 61c is formed having a curved surface portion 61d substantially in the form of a concave mirror that can easily cause cavitation during cleaning operation.

Further, the cleaning probe 61 has vibration nodes that are arranged in a plurality of positions along its axis. It is fitted with rubber rings 62, which are arranged corresponding to several vibration nodes. The rings 62 serve to prevent the internal parts of the handle unit 4 from interfering with the cleaning probe 61.

The overall length of the cleaning probe 61 of the present embodiment is equal to an integral multiple of the half-wave length of the ultrasonic vibration. The probe 61 is designed so that the cavitation portion 61c at its distal end is situated somewhat short of the proximal end portion of the jaw 23 of the tip working portion 14 when the probe 61 is joined to the handle unit 4, as shown in FIG. 8.

As shown in FIG. 8, moreover, the ultrasonic treatment system of the present embodiment is provided with a cleaning tank 63 in which the distal end portion of the insert section 13 of the handle unit 4 can be immersed in the cleaning fluid. The tank 63 has a cleaning tank body 63a in the form of a bottomed cylinder.

An inlet portion 64 of the insert section 13 of the ultrasonic instrument 1 is located at an opening end portion of the cleaning tank body 63a. The inlet portion 64 is fitted with a gasket 65 that is formed of an elastic material such as rubber. Further, the cleaning tank 63 is filled with the cleaning fluid. The gasket 65 serves to prevent the fluid from leaking out as the insert section 13 of the handle unit 4 of the instrument 1 is inserted into the inlet portion 64.

A stabilizer 66 for fixing the insert section 13 of the ultrasonic instrument 1 is attached to the inner peripheral surface of the inlet portion 64 of the cleaning tank 63. A passage hole 66a for the insert section 13 penetrates the center portion of the stabilizer 66. Further, a plurality of cleaning fluid circulation holes 66b are formed in the peripheral edge portion of the stabilizer 66.

According to the present embodiment, as shown in FIG. 8, the cleaning probe 61, in place of the treatment probe 33 of the ultrasonic instrument 1 of the first embodiment, is joined to the handle unit 4. The transducer of the transducer unit 2 is driven in this state, the insert section 13 of the handle unit 4 of the ultrasonic instrument 1 is inserted into the cleaning tank 63 through the inlet portion 64. As ultrasonic waves are then generated by means of the transducer of the transducer unit 2, ultrasonic vibration from the transducer is transmitted to the cleaning probe 61 through the horn 7. Thereupon, the curved surface portion 61d of the cavitation portion 61c at the distal end of the cleaning probe 61 causes cavitation, whereby the tip working portion 14 of the ultrasonic instrument 1 can be efficiently cleaned just after surgical operation.

According to the present embodiment arranged in this manner, therefore, the handle unit 4 is assembled in a manner such that the cleaning probe 61, in place of the treatment probe 33 of the ultrasonic instrument 1, is joined to the transducer unit 2. By doing this, the distal end portion of the probe 61 can be located in an optimum position for cleaning.

Further, the tip working portion 14 can be cleaned by ultrasonic oscillation in a manner such that the cleaning tank 63 is filled with the cleaning fluid and that the distal end portion of the insert section 13 of the handle unit 4 is inserted into the tank 63.

The cleaning tank 63 may be replaced with any other vessel that is filled with the cleaning fluid. In either of the first and second embodiments, moreover, a cleaning mode may be set in the body (not shown) of the ultrasonic treatment system for surgical operation. If a switch is turned on after the mode is changed, in this case, ultrasonic waves can be automatically generated for an effective time for cleaning.

It is to be understood that the present invention is not limited to the embodiments described above, and that various changes and modifications may be effected therein without departing from the scope or spirit of the invention.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment apparatus comprising:
an ultrasonic instrument which has ultrasonic vibration generating means for generating ultrasonic vibration and treats organic tissue with the ultrasonic vibration generated by the ultrasonic vibration generating means;
an ultrasonic cleaning means for cleaning a medical appliance by means of the ultrasonic vibration; and
an electric information output means for delivering electric information for generating the ultrasonic vibration to the ultrasonic vibration generating means and/or the ultrasonic cleaning means.

2. An ultrasonic treatment apparatus comprising:
an application for ultrasonic treatment; and
a cleaning unit for cleaning an operating apparatus by utilizing ultrasonic vibration,
the application comprising a transducer, a power source which outputs electric information to cause the transducer to generate ultrasonic vibration, and a probe which has a proximal end portion removably attached to the transducer and a distal end portion fitted with a tip treatment portion for treating organic tissue and transmits the ultrasonic vibration from the transducer to the tip treatment portion.

3. An ultrasonic treatment apparatus according to claim 2, wherein the cleaning unit comprises a cleaning tank capable of being attached to and detached from the transducer and a probe for transmitting the ultrasonic vibration.

4. An ultrasonic treatment apparatus according to claim 2, wherein the application comprises a sheath unit which covers any part of the probe other than the tip treatment portion and has a control section on the hand side, and the cleaning unit comprises an ultrasonic cleaning probe capable of being attached to and detached from the sheath unit and the transducer.

5. An ultrasonic treatment apparatus according to claim 4, wherein a distal end portion of the sheath unit is provided with a tip working portion capable of being opened and closed with respect to the distal end portion of the probe, and the control section has a control handle for opening and closing the tip working portion.

6. An ultrasonic treatment apparatus according to claim 2, wherein the cleaning unit can be attached to the distal end portion of the operating apparatus having an elongate insert section.
